SEQUENCE LISTING (32 sequences)

SEQ ID NO: 1 - protein, 269 aa, synthetic construct
MKSHEIETSHGRIAIRESRGNGTPLLMIHGNSSAGAIFAPQLEGEIGRNWRVIAPDLPGHGQSGNALDPDRSYSMEGYADVMTEVLAKLGISDAVVFGWSLGGHIGIEMISRFPGMRGLMITGTPPVAREEVGQGFRSGPDMALAGQEAFSDRDVESYARSTCGEPFEEQLLEIVARTDGRARRIMFEKFAAGTGGNQRDIVAEARLPIAVVNGRDEPFVELDFVSKVRFGNLWEGRTHVIDGAGHAPFRETPAIFDDYLQRFMRDCTA

SEQ ID NO: 2 - DNA, 810 bp, synthetic construct
  source 1..810: mol_type = other DNA
atgaaatcccatgaaatcgagaccagtcacggtcgcattgccatccgtgaaagcaggggcaacggaacgcccttgctgatgatccatggcaactccagcgcaggcgccatcttcgcgccccagcttgaaggcgaaatcggtagaaactggcgggtaatcgcgccggatttgccaggccatggccagtccggcaacgcccttgatccggatcgcagctattccatggaaggctatgccgacgtgatgacggaagtgctggcgaagctcggcatttcggatgcggtcgtttttggctggtcgcttggcggccacatcggcatcgagatgatttcacgctttcccggcatgcgcggcctgatgattaccggcacgccgcctgtggcgcgtgaggaagtggggcaggggttcaggagcggcccggatatggcgcttgccgggcaggaggcattttccgaccgcgatgtcgaatcctatgcgcgcagtacctgcggcgaaccgtttgaagagcagttgcttgagattgtcgcccgcacggatgggcgcgcgcggcgcatcatgttcgaaaaatttgcagccggaaccggtggcaaccagcgcgatatcgttgccgaagcaagactcccgattgcagtcgtcaacggtcgtgacgagccgtttgttgaactggatttcgtctcgaaagtccgctttggcaatctctgggaaggcagaacccatgtcatcgacggggcagggcatgcgcctttccgcgagacaccagctatcttcgatgattatctgcagcgtttcatgcgcgactgcacggcctga

SEQ ID NO: 3 - protein, 229 aa, synthetic construct
MTSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYG

SEQ ID NO: 4 - DNA, 690 bp, synthetic construct
  source 1..690: mol_type = other DNA
atgacaagcttacctacgccctcagcaattacgacttcggcgttagagtatgcattccatactcccgctcgcaatctaacgaaatctcgcggaaaaaatattcatcgttatgtcagtgtaaagatgagtaagaggattacggtagaatctactctagagtgtgatgcctgctatcactttgattttgagccaagtattgttcgcttttgcgctcaaccgattcgatttttatattatctcaatggtcagtctcactcctatgttcctgactttctagttcaatttgataccaacgagtttgttctatatgaagtaaagtcagcttatgctaagaacaaacctgattttgatgttgaatgggaggcgaaagtaaaagcagcaactgaactagggctagaattggagcttgttgaagagagtgatattagggatacggttgtattaaataatcttaagcgcatgcatcgttatgcttcgaaagatgagctgaataacgtacataactctctcttaaaaataataaagtacaatggcgcccaatctgcaagatgcttgggagaacagttgggtttaaaaggccgaactgttttaccaattttgtgcgatttgctgtcaaggtgtttactcgatacacgtttggataagcctctatctcttgaatctcgatttgagttggccagttatggctaa

SEQ ID NO: 5 - protein, 603 aa, synthetic construct
MAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 6 - DNA, 1812 bp, synthetic construct
  source 1..1812: mol_type = other DNA
atggctaagaaagggttctcgagtttccatagaaaagcagtctcttcccaagatacgcttgaatcgatagagcttgtctctagcgctaattgtctagaaagtgttacgtatcaagatatatcagcatttcccgaaacaattgcggtagagattaatttccgattaagcattcttcgctttttagcgcgaaagtgcgaaaccattgtggccaaatcaattgaaccacatcgtgtagagctacagcaaaactatagtagaaaaatacccagtgcaataacgatatatcgatggtggcttgcttttcgaaaatcagactacaaccccattagcttagcacctaatatcaaggatagaggtaatagagagacaaaagtgtcaacagttgttgattctattatggaacaggcagttgaaagagttatatctggacgaaaagtcaatgttagctctgcatataaacgtgttcgacgaaaagttcgtcaatacaatctaactcatggaacgaaatacacgtatcctaagtacgaatctgtaagaaagcgagtaaaaaagaaaaccccatttgagttattagccgcagggaaaggggagagagtagctaagagagagtttcgccgaatgggaaaaaagatcctcacgtctagcgtgctagagagggttgaaatagatcacactgtcgttgacctttttgcagtacacgaagagtatcgaattccattgggccgaccttggcttactcaattggttgattgttacagtaaagctgtaatcggtttttatttaggtttcgagcctcctagctatgtgtcggtttcccttgcacttaagaatgcaatacaacgcaaagatgacttaatctcctcgtatgaatcgatcgagaatgaatggctatgttatgggatcccagacctactcgtaactgataatggtaaagagtttttgtcgaaagcatttgatcaagcatgtgaatcactattgatcaatgtgcatcaaaataaagttgagacgcccgacaacaaacctcatgttgaacgtaactacgggactattaatacttctctgttagacgatttacctgggaaatccttcagccagtaccttcaaagagaagggtacgactctgtgggagaagctacccttacactcaatgagattagagaaatttacttaatttggttggtggatatttatcataaaaaacccaatcagagaggcactaattgtcctaatgttgcgtggaaaaagggttgtcaagaatgggaaccagaggagttctctggttctaaagacgaattagactttaaatttgctattgttgattacaaacaacttactaaagtagggataactgtctacaaagaactgagttatagcaatgaccgtttagctgaatatagagggaagaaaggaaaccataaagttcagttcaagtataaccctgagtgtatggcagttatttgggtgttggatgaggatatgaatgagtactttacagttaatgcgattgactacgaatatgcaagtagagtatcactttggcaacataaatataacatgaaatatcaagcagaactaaattcagcagaatatgatgaagacaaggaaattgatgcagaaataaaaattgaagaaatcgcagatcgttcaattgttaagactaacaaaatcagagctcggaggcgtggcgctaggcatcaagagaatagcgcaagggctaagtcaatcagtaatgcgaacccggcctcgatacaaaaacatgaagatgaaatcgttagtgcagataatgacgattgggatattgattatgtctga

SEQ ID NO: 7 - protein, 330 aa, synthetic construct
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGWEDYLRHSTPRVSKPGRNKNFFE

SEQ ID NO: 8 - DNA, 993 bp, synthetic construct
  source 1..993: mol_type = other DNA
atgagtgaaacgcgtgaggctcgtatatcaagagctaaaagggcatttgtatccacaccgagcgttaggaaaatcttgagttacatggatagatgtagagacctatcagacctagagtctgagcctacatgcatgatggtctatggtgcttcgggtgtaggtaaaacgaccgtcatcaagaaatacttaaatcaaaacagaagagagtccgaagccgggggcgatataataccggttttgcatattgagttgccagacaatgcgaagccagtagatgcagcaagggaattgctggttgaaatgggtgacccgctagcactttatgaaactgacttagctagattgacgaaaagactgactgaattaatccctgcggtcggcgtgaagctgattattatcgatgagttccaacatttggtggaagaaaggtcaaatcgggttcttacccaagtaggtaattggctaaaaatgatacttaacaaaacgaaatgtccaattgttatatttggtatgccatactcaaaagttgtactgcaagcaaactcgcaacttcacgggcgattttccattcaggttgaactgcgcccctttagctaccagggaggtagaggtgtatttaaaacttttttggaataccttgataaagccctaccttttgaaaaacaggctggcttagccaacgaaagtttgcagaaaaaattgtatgcattctctcagggaaacatgcgttcgttgagaaaccttatttatcaagcatctatcgaagcaattgataatcagcatgagacgataaccgaagaagatttcgtttttgcatcgaagttgacatcgggcgataaacccaactcatggaaaaatccttttgaggagggtgttgaggtaacagaagatatgttacgaccgccaccaaaagatattggttgggaagactatttgagacattcaaccccgagagtgagtaaaccaggtagaaataaaaactttttcgaataa

SEQ ID NO: 9 - DNA, 54 bp, synthetic construct
  source 1..54: mol_type = other DNA
agaaggagagctagcgccggcatgaaatcccatgaaatcgagaccagtcacggt

SEQ ID NO: 10 - DNA, 47 bp, synthetic construct
  source 1..47: mol_type = other DNA
ttaactcgagctcgagtcaatgatgatgatgatgatgggccgtgcag

SEQ ID NO: 11 - DNA, 19 bp, synthetic construct
  source 1..19: mol_type = other DNA
caactggtcacgctgatcg

SEQ ID NO: 12 - DNA, 20 bp, synthetic construct
  source 1..20: mol_type = other DNA
ctgttcaacctggtccagcc

SEQ ID NO: 13 - DNA, 21 bp, synthetic construct
  source 1..21: mol_type = other DNA
gccatgctaactcgtccaaac

SEQ ID NO: 14 - DNA, 20 bp, synthetic construct
  source 1..20: mol_type = other DNA
gcgtatgaagagcggtgagc

SEQ ID NO: 15 - DNA, 20 bp, synthetic construct
  source 1..20: mol_type = other DNA
gtggaattactggcagcgac

SEQ ID NO: 16 - DNA, 23 bp, synthetic construct
  source 1..23: mol_type = other DNA
cggtggtatattcccttctgatg

SEQ ID NO: 17 - DNA, 19 bp, synthetic construct
  source 1..19: mol_type = other DNA
ctgcatgtacgtgccttcc

SEQ ID NO: 18 - DNA, 22 bp, synthetic construct
  source 1..22: mol_type = other DNA
gctaccgaagggactattcctc

SEQ ID NO: 19 - DNA, 24 bp, synthetic construct
  source 1..24: mol_type = other DNA
cagagatggaaattaccctgcaag

SEQ ID NO: 20 - DNA, 19 bp, synthetic construct
  source 1..19: mol_type = other DNA
cgtttgccgttcaacaacg

SEQ ID NO: 21 - DNA, 23 bp, synthetic construct
  source 1..23: mol_type = other DNA
ggtgcaagtatcatcaccacttc

SEQ ID NO: 22 - DNA, 24 bp, synthetic construct
  source 1..24: mol_type = other DNA
gtcgattacgagacgttgatgaag

SEQ ID NO: 23 - DNA, 24 bp, synthetic construct
  source 1..24: mol_type = other DNA
ctggtatcgtaaaatggttcaacg

SEQ ID NO: 24 - DNA, 26 bp, synthetic construct
  source 1..26: mol_type = other DNA
atgccgcagaaatatagattactttc

SEQ ID NO: 25 - DNA, 20 bp, synthetic construct
  source 1..20: mol_type = other DNA
tcttccgcattaccctggtg

SEQ ID NO: 26 - DNA, 22 bp, synthetic construct
  source 1..22: mol_type = other DNA
tacactgaactctctggctcac

SEQ ID NO: 27 - DNA, 24 bp, synthetic construct
  source 1..24: mol_type = other DNA
cactgtaatgataggcaattcagg

SEQ ID NO: 28 - DNA, 21 bp, synthetic construct
  source 1..21: mol_type = other DNA
atgaagcagcataaggcgatg

SEQ ID NO: 29 - DNA, 19 bp, synthetic construct
  source 1..19: mol_type = other DNA
gaagcgcaacaggcatctg

SEQ ID NO: 30 - DNA, 24 bp, synthetic construct
  source 1..24: mol_type = other DNA
gtgagatccaaactgatgcaggtt

SEQ ID NO: 31 - DNA, 24 bp, synthetic construct
  source 1..24: mol_type = other DNA
cgtcatcttgataaaggaacgcgc

SEQ ID NO: 32 - DNA, 22 bp, synthetic construct
  source 1..22: mol_type = other DNA
agggaggcaagacatgctgaca

(19) United States
(12) Patent Application Publication
(10) Pub. No.: US 2026/0275361 A1
(43) Pub. Date: Sep. 17, 2026

(54) RECOMBINANT STRAIN OF N-ACYL-HOMOSERINE LACTONASE AIO6 WITHOUT RESISTANCE SELECTION MARKER AND USE THEREOF

(71) Applicant: INSTITUTE OF FEED RESEARCH OF CHINESE ACADEMY OF AGRICULTURAL SCIENCES, Beijing (CN)

(72) Inventors: Zhigang ZHOU, Beijing (CN); Yalin YANG, Beijing (CN); Rui XIA, Beijing (CN); Ya JIN, Beijing (CN); Yuanyuan YAO, Beijing (CN); Chao RAN, Beijing (CN); Zhen ZHANG, Beijing (CN); Qianwen DING, Beijing (CN); Nan WANG, Beijing (CN); Xiaoqing XU, Beijing (CN); Gangqiang LI, Beijing (CN)

(73) Assignee: INSTITUTE OF FEED RESEARCH OF CHINESE ACADEMY OF AGRICULTURAL SCIENCES, Beijing (CN)

(21) Appl. No.: 19/567,456

(22) Filed: Mar. 16, 2026

(30) Foreign Application Priority Data
Mar. 17, 2025 (CN) 202510307352.X

(51) Int. Cl. C12N 15/70; C12N 9/18

(52) U.S. Cl. CPC C12N 15/70; C12N 9/18; C12Y 301/01081

(57) ABSTRACT

A recombinant strain of N-acyl-homoserine lactonase AIO6 without resistance selection marker and its application are provided. The method for constructing a recombinant Escherichia coli strain without resistance selection marker includes introducing the coding gene of N-acyl-homoserine lactonase AIO6 into recipient Escherichia coli, followed by screening without antibiotic resistance markers to obtain the recombinant E. coli. The construction of this recombinant strain of N-acyl-homoserine lactonase AIO6 without resistance selection marker significantly enhances the application of AIO6 in production. Moreover, compared to previously reported AIO6 strains, the N-acyl-homoserine lactonase activity of this strain has been further improved.

### CROSS REFERENCE TO THE RELATED APPLICATIONS

This application is based upon and claims priority to Chinese Patent Application No. 202510307352.X, filed on Mar. 17, 2025, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named KJ0133S_SequenceListing.xml, created on Mar. 6, 2026, and is 34,823 bytes in size.

### TECHNICAL FIELD

This disclosure pertains to the field of microbiology and enzyme technology, specifically to a recombinant strain of N-acyl-homoserine lactonase AIO6 without antibiotic resistance screening markers and use thereof.

### BACKGROUND

As the most crucial screening marker, resistance genes play a significant role in rapidly obtaining transformants. However, with the development of biotechnology and the commercialization of genetically modified products, biosafety has garnered widespread public attention, particularly concerning the safety of screening marker genes introduced into species. These screening marker genes may spread among populations or transfer to other populations through certain pathways, posing potential hazards to the ecological environment and species diversity. Moreover, there are concerns that genetically modified products and their derivatives containing screening markers may harm human or animal health. Therefore, utilizing genetic transformation techniques without antibiotic resistance screening markers to obtain desired products is currently a research hotspot.

Since the 1950s, antibiotics have been widely used as feed additives in the breeding industry due to their functions in promoting growth and preventing animal diseases. However, long-term misuse of antibiotics can lead to increased bacterial resistance, harm animal health, damage the ecological environment, and ultimately threaten human health. Therefore, restricting and banning the use of antibiotics has become an inevitable choice for the green development of the breeding industry and ensuring food safety.

Quorum sensing (QS) is a phenomenon in which microorganisms communicate through the synthesis, secretion, and response to diffusible small-molecule signaling molecules. It is one of the key points in the regulation of pathogen virulence. Based on the role of microbial QS in the pathogenic mechanisms of bacterial regulation, the quorum quenching (QQ) method has emerged. This method interferes with quorum sensing by quenching the signaling molecules of pathogenic bacteria and preventing their effective accumulation, thereby preventing diseases. The goal of the quorum quenching strategy is not to kill pathogens or limit cell growth but to turn off the expression of pathogenic genes. Therefore, it does not induce drug resistance in pathogens and has no side effects on the external environment, boasting a series of advantages such as simple and convenient operation, low cost, environmental friendliness, high efficiency, and short cycle times. Quorum quenching has been increasingly proven by research to be an effective new strategy for disease prevention and control, becoming a frontier and hotspot in research related to microbial diseases and their prevention and treatment technologies.

N-acyl-homoserine lactones (AHLs) are a class of quorum sensing signaling molecules widely present in Gram-negative bacteria. They participate in regulating the pathogenic processes of various pathogens and are thus often considered potential targets for alleviating or controlling bacterial diseases. N-acyl-homoserine lactonases can degrade the signaling molecule AHL, blocking the bacterial quorum sensing signaling pathway and preventing bacteria from initiating the expression of virulence factors, leading to a reduction or loss of bacterial pathogenicity and virulence. They have been successfully used in the prevention and treatment of bacterial diseases. Research has shown that the use of N-acyl-homoserine lactonases can not only reduce the virulence of pathogens and inhibit biofilm formation but also significantly improve the composition of intestinal microbiota and enhance host disease resistance. In aquaculture, feeding N-acyl-homoserine lactonases derived from Bacillus significantly reduces the adhesiveness of pathogenic bacteria and improves the disease resistance of zebrafish, koi, and goldfish. The N-acyl-homoserine lactonase AiiA derived from Bacillus licheniformis can inhibit biofilm formation by Vibrio spp. and reduce their colonization in the shrimp intestine, as well as enhance shrimp immunity. In poultry farming, N-acyl-homoserine lactonase AIO6 can improve feed conversion efficiency and daily weight gain in broiler chickens. The daily weight gain of broiler chickens in the experimental group increased by 3.37%, and the feed-to-gain ratio in the group fed with AIO6 is significantly lower by 0.04 compared to the control group, with the feed conversion efficiency in the experimental group of broiler chickens increasing by 2.30%. Adding quorum sensing quenching enzymes, glucose oxidase, and lactic acid bacteria to the basal diet significantly increased the growth performance of broiler chickens during the early growth stage (1-21 days old), significantly elevated serum IL-6 levels, improved serum CAT and GSH-Px activities, as well as liver SOD activity, and reduced fecal uric acid levels. However, N-acyl-homoserine lactonase recombinant strains are usually obtained through methods involving resistance screening markers, which have certain limitations in production applications and may pose significant threats to the public environment and health. Therefore, obtaining N-acyl-homoserine lactonase recombinant strains without antibiotic resistance screening markers is of utmost importance.

### SUMMARY

The problem to be solved by this disclosure is the current lack of AIO6 recombinant Escherichia coli strains without antibiotic resistance screening markers.

In order to address the problem, this disclosure provides a recombinant Escherichia coli strain without antibiotic resistance screening markers for AIO6, as well as a method for constructing recombinant Escherichia coli strains without antibiotic resistance screening markers.

This disclosure has obtained recombinant Escherichia coli strains without antibiotic resistance screening markers, which involves introducing the coding gene of N-acyl-homoserine lactonase AIO6 into recipient Escherichia coli, followed by screening without antibiotic resistance markers to obtain recombinant Escherichia coli. The introduction process includes the following steps:
1) introducing a vector containing N-acyl-homoserine lactonase AIO6 and a vector containing the coding genes of transposase proteins TnsA, TnsB, and TnsC into recipient Escherichia coli, and culturing to obtain transformant A;
2) introducing a multi-target gene editing plasmid into the transformant A described in step 1), and culturing to obtain transformant B;
3) conducting primary screening of the transformant B described in step 2) to select strain C with high transposition efficiency and/or high enzyme activity of N-acyl-homoserine lactonase AIO6; and
4) introducing the pCutamp vector into the strain C to stop the transposition process, and screening using an antibiotic plate. The strain that can grow on a sucrose medium but cannot grow on the antibiotic plate is the recombinant Escherichia coli strain without antibiotic resistance screening markers.

In the above method, the amino acid sequence of N-acyl-homoserine lactonase AIO6 is SEQ ID NO: 1, and the nucleotide sequence of the coding gene is SEQ ID NO: 2.

In this method, the vector containing N-acyl-homoserine lactonase AIO6 can specifically be the pRE57I-AIO6 vector. The structural description of the pRE57I-AIO6 vector is as follows: a DNA fragment with the sequence of SEQ ID NO: 2 is inserted between the NaeI and XhoI restriction sites of the starting vector pRE57I, while keeping other sequences of the pRE57I vector unchanged, resulting in a recombinant vector. The pRE57I-AIO6 vector can express N-acyl-homoserine lactonase AIO6, with its amino acid sequence being SEQ ID NO: 1.

In the method, the vector containing the coding genes of TnsABC proteins can be the vector pTnsABC.

The amino acid sequence of TnsA is SEQ ID NO: 3, and the nucleotide sequence of its coding gene is SEQ ID NO: 4. The amino acid sequence of TnsB is SEQ ID NO: 5, and the nucleotide sequence of its coding gene is SEQ ID NO: 6. The amino acid sequence of TnsC is SEQ ID NO: 7, and the nucleotide of its coding gene is SEQ ID NO: 8.

In the method, the multi-target gene editing plasmid is pTetQcas-8+IS186.

The target sites are located at positions 16413-16444, 150770-150801, 426765-426796, 569521-569552, 1794404-1794435, 2688153-2688184, 2415243-2415274, 745115-745146, 2987107-2987138, 1269372-1269403, 3551707-3551738, 1847019-1847050, and 4107738-4107769 of the Escherichia coli genome sequence (Genbank: GCF_000833145.1, updated on Feb. 5, 2015).

The antibiotic resistance screening markers can be ampicillin resistance markers, kanamycin resistance markers, and/or streptomycin resistance markers.

In step 4) of the method, when screening using an antibiotic plate, the antibiotic plates used contain ampicillin, kanamycin, and streptomycin, respectively.

In this context, the transposition efficiency can be evaluated by detecting the number of target gene insertions into transposition sites via PCR.

The enzyme activity of N-acyl-homoserine lactonase AIO6 is defined as follows: under conditions of 30° C. and pH 6.5, the amount of enzyme required to hydrolyze 1 nmoL of N-acyl-homoserine lactone from a 50 μg/mL N-acyl-homoserine lactone solution per minute is considered as one unit of N-acyl-homoserine lactonase activity, denoted as U.

This disclosure also provides the method for use in any one of the following applications:
P1. producing N-acyl-homoserine lactonase;
P2. increasing the yield of N-acyl-homoserine lactonase; and
P3. preparing a recombinant strain producing N-acyl-homoserine lactonase without antibiotic resistance screening markers.

The recombinant Escherichia coli strain constructed by the method also falls within the scope of protection claimed by this disclosure. The recombinant Escherichia coli strain can express N-acyl-homoserine lactonase AIO6.

This disclosure also provides the Escherichia coli strain for use in anyone of the following applications:
P1. producing N-acyl-homoserine lactonase; and
P2. increasing the yield of N-acyl-homoserine lactonase.

This disclosure also provides a method for producing N-acyl-homoserine lactonase, which involves culturing the recombinant Escherichia coli strain to obtain a fermentation product and then isolating N-acyl-homoserine lactonase from the fermentation product.

Recombinant strains of N-acyl-homoserine lactonase typically contain antibiotic resistance screening marker genes, which have certain limitations in production applications and significantly reduce their economic value. This disclosure has obtained a recombinant strain of N-acyl-homoserine lactonase AIO6 without antibiotic resistance screening markers. The construction of this antibiotic-free strain greatly enhances the application of AIO6 in production, and the activity of this strain has also been further improved compared to previously reported AIO6 strains.

### BRIEF DESCRIPTION OF THE DRAWINGS

FIGS. 1A-ID show the PCR identification results of the transposition efficiency of AIO6 transposon strains at sites site1 to site4. Among them, a represents the insertion of AIO6 at site1; b represents the insertion of AIO6 at site2; c represents the insertion of AIO6 at site3; d represents the insertion of AIO6 at site4; M: DNA Marker. Lanes 1-24 correspond to transposon strains numbered 1-24.

FIGS. 2A-2D show the PCR identification results of the transposition efficiency of AIO6 transposon strains at sites site5 to site8. a represents the insertion of AIO6 at site5; b represents the insertion of AIO6 at site6; c represents the insertion of AIO6 at site7; d represents the insertion of AIO6 at site8; M: DNA Marker. Lanes 1-24 correspond to transposon strains numbered 1-24.

FIGS. 3A-3E show the PCR identification results of the transposition efficiency of AIO6 transposon strains at sites IS186-1 to IS186-5. a represents the insertion of AIO6 at IS186-1; b represents the insertion of AIO6 at IS186-2; c represents the insertion of AIO6 at IS186-3; d represents the insertion of AIO6 at IS186-4; e represents the insertion of AIO6 at IS186-5; M: DNA Marker. Lanes 1-24 correspond to transposon strains numbered 1-24.

FIGS. 4A-4C show the SDS-PAGE results of the induced expression of the target protein in AIO6 transposon strains. Among them, a represents the whole-cell protein of AIO6 transposon strains after induction; b represents the insoluble protein of AIO6 transposon strains treated with BugBuster; c represents the soluble protein of AIO6 transposon strains treated with BugBuster. M: Protein Marker. AIO6 represents the wild-type AIO6 strain, and 1, 2, 11, 12, 13, 19, 22, 23, and 24 are the corresponding strain numbers.

FIGS. 5A-5B show the elimination status of the transposon plasmid in AIO6 transposon strains. Among them, a represents the growth of strain 12 on LB medium containing ampicillin (Amp), kanamycin (Km), streptomycin (Strep), and 15% sucrose, respectively; b represents the growth of strain 13 on LB medium containing ampicillin (Amp), kanamycin (Km), streptomycin (Strep), and 15% sucrose, respectively.

FIGS. 6A-6B show the elimination status of the pCutamp plasmid in AIO6 transposon strains. Among them, a represents the growth of transposon strain 12 on LB medium containing apramycin (Apra+LB) and LB medium (LB); b represents the growth of transposon strain 13 on LB medium containing apramycin (Apra+LB) and LB medium (LB).

FIG. 7 shows the SDS-PAGE results of the induced expression of the target protein in AIO6 plasmid-free multi-copy integrated strains. Lane 1 represents the total protein of the wild-type AIO6, lane 2 represents the soluble protein of the wild-type AIO6, lane 3 represents the total protein of strain 12-1, lane 4 represents the soluble protein of strain 12-1, lane 5 represents the total protein of strain 12-3, lane 6 represents the soluble protein of strain 12-3, lane 7 represents the total protein of strain 13-2, lane 8 represents the soluble protein of strain 13-2, lane 9 represents the total protein of strain 13-36, and lane 10 represents the soluble protein of strain 13-36.

### DETAILED DESCRIPTION OF THE EMBODIMENTS

The present disclosure will be further described in detail below in conjunction with specific embodiments. The provided examples are solely for the purpose of illustrating the disclosure and are not intended to limit the scope of the disclosure. The examples provided below can serve as a guide for further improvement by professionals in the technical field and do not constitute any restriction on the disclosure in any way.

Unless otherwise specified, the experimental methods in the following examples are conventional methods, carried out in accordance with the techniques or conditions described in the literature within the field or according to the product instructions. Unless otherwise specified, the materials, reagents, etc., used in the following examples can be obtained through commercial channels.

Unless otherwise specified, for quantitative experiments in the following examples, three replicate experiments are set up, and the results are taken as the average value.

The pRE57I-GNac in the following examples is purchased from GenSmart (Cat.NO.: MC_0101251).

The pTet-tns in the following examples is purchased from GenSmart (Cat.NO.: MC_0101242).

The pTetQcas-8+IS186 in the following examples is purchased from GenSmart (Cat.NO.: MC_0101250).

The pCutamp in the following examples is purchased from GenSmart (Cat.NO.: MC_0101104).

The pET28a in the following examples is purchased from Beijing Zoman Biotechnology (ZK159).

The following examples use SPSS 11.5 statistical software for data processing. The experimental results are presented as the mean±standard deviation. A One-way ANOVA test is used. A P-value of less than 0.05 (*) indicates a significant difference, a P-value of less than 0.01 (**) indicates a highly significant difference, and a P-value of less than 0.001 (***) indicates an extremely significant difference.

### Example 1: Transposition Experiment in Escherichia coli

### I. Construction Method of Recombinant Vectors

1. Construction of the Recombinant Vector pRE57I-AIO6

The target fragment AIO6 (with the nucleotide sequence as SEQ ID NO: 2) is obtained using the primer pair AIO6-F: 5′-AGAAGGAGAGCTAGCgccggcATGAAATCCCATGAAATCGAGACCAGTCACGGT-3′ as shown in SEQ ID NO: 9 and AIO6-R: 5′-TTAACTCGAGctcgagTCAATGATGATGATGATGATGGGCCGTGCAG-3′ as shown in SEQ ID NO: 10. This fragment is then cloned into the pRE57I vector using the NaeI and XhoI double-digestion restriction sites.

The structural description of the pRE57I-AIO6 vector is as follows: A DNA fragment with the sequence of SEQ ID NO: 2 is inserted between the NaeI and XhoI restriction sites of the starting vector pRE57I-GNac, while the other sequences of the pRE57I vector remained unchanged, resulting in the recombinant vector. The pRE57I-AIO6 vector can express N-acyl homoserine lactonase AIO6, with its amino acid sequence being SEQ ID NO: 1.

### II. Transposition Experiment

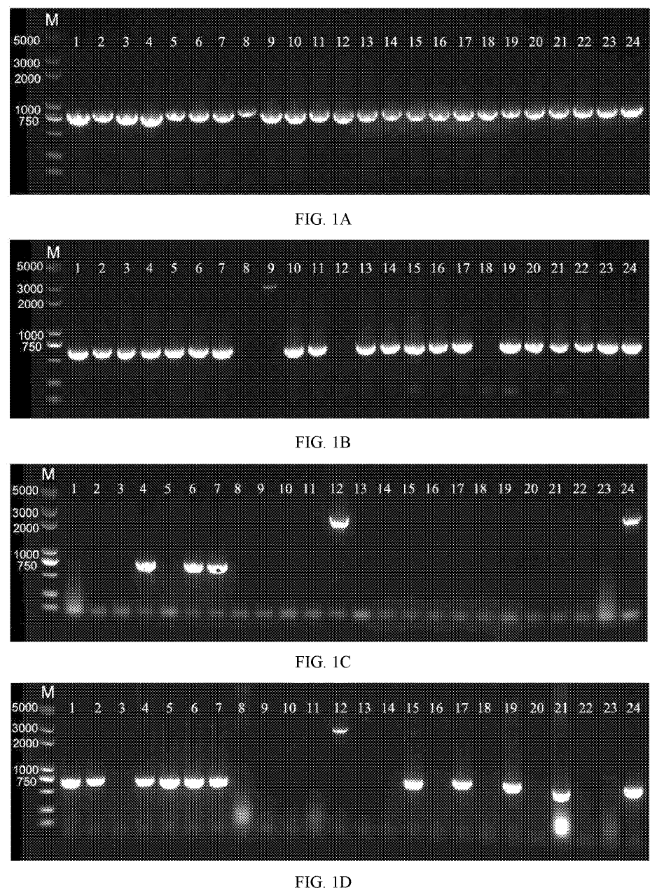

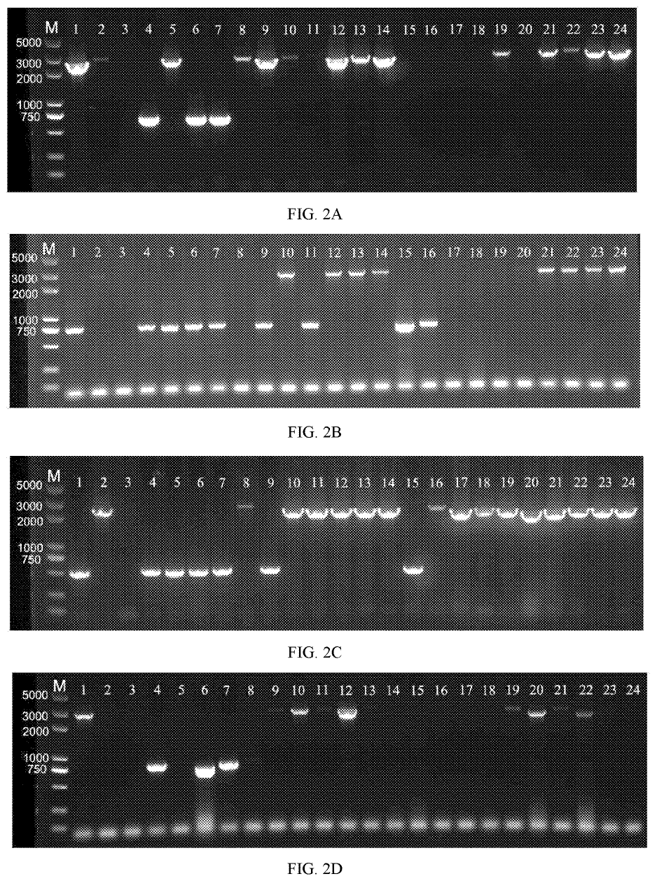

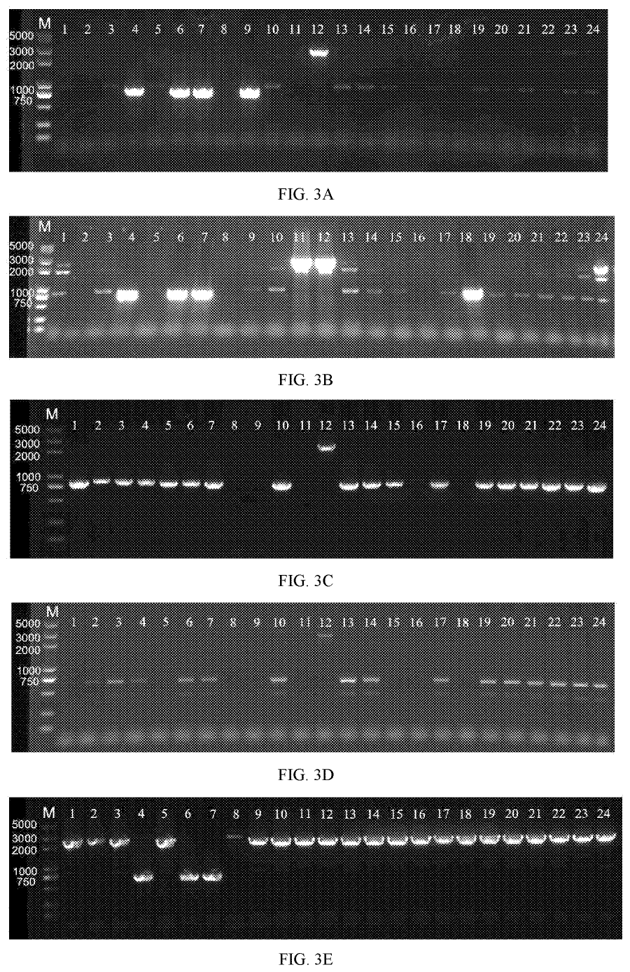

1. The pRE57I-AIO6 and pTet-tns vectors are co-transformed into Escherichia coli competent cells (Vazyme, C502-03). Transformants A are screened using LB agar plates containing 100 μg/mL ampicillin (Yuanye, S17018-25g) and 50 μg/mL kanamycin (Lablead, 0408-10G).
2. The positive clone transformants A screened in step 1 are prepared into competent cells. The pTetQcas-8+IS186 vector is then transformed into these competent cells. After recovery, the bacterial culture is inoculated onto LB agar plates containing 100 μg/mL ampicillin, 50 μg/mL kanamycin, and 50 μg/mL streptomycin (ThermoFisher Scientific, 15140122). After incubation at 37° C. for 16 hours, positive clone transformants B are obtained.
3. Several hundred positive clone transformants B obtained in step 2 are scraped from the plates and resuspended in fresh LB medium. Subsequently, they are inoculated onto LB agar plates containing triple antibiotics (100 μg/mL ampicillin, 50 μg/mL kanamycin, and 50 μg/mL streptomycin, respectively) and 0.1 μg/mL anhydrotetracycline (Zomanbio, ZS815). The plates are incubated at 37° C. for 16 hours to induce the expression of transposition-related proteins.
4. After the biofilm formation of the bacteria obtained in step 3, they are scraped and resuspended in LB medium. The bacteria are then diluted and re-inoculated onto LB agar plates containing triple antibiotics and 1 μg/mL anhydrotetracycline. The plates are incubated overnight at 37° C.
5. The colonies obtained in step 4 are named as transconjugants 1. Some of the transconjugants 1 are streaked onto LB agar plates containing triple antibiotics and 1 μg/mL anhydrotetracycline and incubated overnight at 37° C. to obtain transconjugants 2. This process is repeated continuously to generate transconjugants 6. Randomly selected strains are subjected to colony PCR to identify the transposition efficiency (as shown in FIGS. 1A-1D, FIGS. 2A-2D, FIGS. 3A-3E, and Table 2). If AIO6 is not inserted into the corresponding site, the band is located between 500 bp and 1000 bp. If AIO6 is inserted into the corresponding site, the band is located between 2000 bp and 3000 bp. The transposition efficiency is determined by the total number of insertions in Table 2; the more insertions, the higher the transposition efficiency. The primers used are listed in Table 1.

### Table 1: Primer Sequences for Identifying Copy Number

Where in Table 1, CAACTGGTCACGCTGATCG is as shown in SEQ ID NO: 11, CTGTTCAACCTGGTCCAGCC is as shown in SEQ ID NO: 12, GCCATGCTAACTCGTCCAAAC is as shown in SEQ ID NO: 13, GCGTATGAAGAGCGGTGAGC is as shown in SEQ ID NO: 14, GTGGAATTACTGGCAGCGAC is as shown in SEQ ID NO: 15, CGGTGGTATATTCCCTTCTGATG is as shown in SEQ ID NO: 16, CTGCATGTACGTGCCTTCC is as shown in SEQ ID NO: 17, GCTACCGAAGGGACTATTCCTC is as shown in SEQ ID NO: 18, CAGAGATGGAAATTACCCTGCAAG is as shown in SEQ ID NO: 19, CGTTTGCCGTTCAACAACG is as shown in SEQ ID NO: 20, GGTGCAAGTATCATCACCACTTC is as shown in SEQ ID NO: 21, GTCGATTACGAGACGTTGATGAAG is as shown in SEQ ID NO: 22, CTGGTATCGTAAAATGGTTCAACG is as shown in SEQ ID NO: 23, ATGCCGCAGAAATATAGATTACTTTC is as shown in SEQ ID NO: 24, TCTTCCGCATTACCCTGGTG is as shown in SEQ ID NO: 25, TACACTGAACTCTCTGGCTCAC is as shown in SEQ ID NO: 26, CACTGTAATGATAGGCAATTCAGG is as shown in SEQ ID NO: 27, ATGAAGCAGCATAAGGCGATG is as shown in SEQ ID NO: 28, GAAGCGCAACAGGCATCTG is as shown in SEQ ID NO: 29, GTGAGATCCAAACTGATGCAGGTT is as shown in SEQ ID NO: 30, CGTCATCTTGATAAAGGAACGCGC is as shown in SEQ ID NO: 31, AGGGAGGCAAGACATGCTGACA is as shown in SEQ ID NO: 32.

| | Primer sequence | |
|---|---|---|
| Sites | (5′ -3′) | Role |
| site1-UF | CAACTGGTCACGCTGATCG | Forward and reverse |
| site1-DR | CTGTTCAACCTGGTCCAGCC | genome-specific primers |
| site2-UF | GCCATGCTAACTCGTCCAAAC | for amplifying the |
| site2-DR | GCGTATGAAGAGCGGTGAGC | transposition products |
| site3-UF | GTGGAATTACTGGCAGCGAC | containing CrRNA-9 to |
| site3-DR | CGGTGGTATATTCCCTTCTGATG | CrRNA-16. |
| site4-UF | CTGCATGTACGTGCCTTCC | |
| site4-DR | GCTACCGAAGGGACTATTCCTC | |
| site5-UF | CAGAGATGGAAATTACCCTGCAAG | |
| site5-DR | CGTTTGCCGTTCAACAACG | |
| site6-UF | GGTGCAAGTATCATCACCACTTC | |
| site6-DR | GTCGATTACGAGACGTTGATGAAG | |
| site7-UF | CTGGTATCGTAAAATGGTTCAACG | |
| site7-DR | ATGCCGCAGAAATATAGATTACTTTC | |
| site8-UF | TCTTCCGCATTACCCTGGTG | |
| site8-DR | TACACTGAACTCTCTGGCTCAC | |
| IS186F | CACTGTAATGATAGGCAATTCAGG | Forward and reverse |
| IS186-1R | ATGAAGCAGCATAAGGCGATG | genome-specific primers |
| IS186-2R | GAAGCGCAACAGGCATCTG | for amplifying |
| IS186-3R | GTGAGATCCAAACTGATGCAGGTT | transposition products |
| IS186-4R | CGTCATCTTGATAAAGGAACGCGC | containing IS186. |
| IS186-5R | AGGGAGGCAAGACATGCTGACA | |

6. By comparing the PCR identification results, it is confirmed that AIO6 is inserted into different sites during the transposition process. The detailed information on the insertion sites is shown in Table 2.

TABLE 2
Identification of Transposition Efficiency of AIO6

| | Strain Identification Number | | | | | | | | | | | | | | | | | | | | | | | |
|---|---|---|---|---|---|---|---|---|---|---|---|---|---|---|---|---|---|---|---|---|---|---|---|---|
| Sites | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
| site 1 | | | | | | | | | | | | | | | | | | | | | | | | |
| site 2 | | | | | | | | | 1 | | | | | | | | | | | | | | | |
| site 3 | | | | | | | | | | | | 1 | | | | | | | | | | | | 1 |
| site 4 | | | | | | | | | | | | 1 | | | | | | | | | | | | |
| site 5 | 1 | 1 | | | 1 | | | 1 | 1 | 1 | | 1 | 1 | 1 | | | | | 1 | | 1 | 1 | 1 | 1 |
| site 6 | | 1 | | | | | | | | 1 | | 1 | 1 | 1 | | | | | | 1 | 1 | 1 | 1 | 1 |
| site 7 | | 1 | | | | | | 1 | | 1 | 1 | 1 | 1 | 1 | | 1 | 1 | 1 | 1 | 1 | 1 | 1 | 1 | 1 |
| site 8 | 1 | | | | | | | | 1 | 1 | 1 | 1 | | | | | | | 1 | 1 | 1 | 1 | | |
| IS186-1 | | | | | | | | | | | | 1 | | | | | | | | | | | 1/0 | |
| IS186-2 | 1/0 | | 1/0 | | | | | | 1/0 | 1/0 | 1 | 1 | 1/0 | 1/0 | | | | | | | 1/0 | 1/0 | 1/0 | 1/0 |
| IS186-3 | | | | | | | | | | | | 1 | | | | | | | | | | | | |
| IS186-4 | | | | | | | | | | | | 1 | | | | | | | | | | | | |
| IS186-5 | 1 | 1 | 1 | | 1 | | | 1 | 1 | 1 | 1 | 1 | 1 | 1 | 1 | 1 | 1 | 1 | 1 | 1 | 1 | 1 | 1 | 1 |
| Sum of | 4 | 4 | 2 | | 2 | | | 3 | 5 | 6 | 4 | 11 | 5 | 5 | 1 | 2 | 2 | 2 | 4 | 4 | 6 | 6 | 6 | 6 |
| inserted | | | | | | | | | | | | | | | | | | | | | | | | |
| fragments | | | | | | | | | | | | | | | | | | | | | | | | |

Note:
“1” indicates successful insertion; “1/0” indicates that there are strains without insertion in the colony.

### III. Induction and Expression of Transposon Strains

1. The transposon strainsl strain with the target gene inserted at the transposition site from step II is inoculated into 20 mL of LB medium containing 100 μg/mL ampicillin, 50 μg/mL kanamycin, and 50 μg/mL streptomycin, and cultured overnight at 37° C. A strain containing pET28a-AIO6 (kanamycin) is used as a control. The strain containing pET28a-AIO6 (kanamycin) is a recombinant strain obtained by introducing the recombinant vector pET28a-AIO6 into Escherichia coli BL21.

The structural description of the recombinant vector pET28a-AIO6 is as follows: a DNA fragment with the sequence of SEQ ID NO: 2 is inserted between the NaeI and XhoI restriction sites of the starting vector pET28a, while the other sequences of the pET28a starting vector remain unchanged, resulting in the recombinant vector.
2. 500 μL of the bacterial culture from step 1 is transferred to 50 mL of LB medium containing the corresponding antibiotics and cultured at 37° C. until the OD₆₀₀ reaches 0.6-0.8.
3. 24 μL of IPTG (final concentration of 0.4 mM) is added, and induction is carried out at 16° C. for 16-18 hours.
4. Three 1-mL aliquots of the induced sample from step 3 are taken: one is used to measure the OD₆₀₀ (200 μL; 4 replicates); one is centrifuged at 12,000 rpm at 4° C., the supernatant is discarded, and the precipitate C (total protein) is collected and stored at −20° C.; one is subjected to a solubility experiment. It is centrifuged at 12,000 rpm at 4° C., the supernatant is discarded, 0.5 mL of BugBuster is added to the precipitate, and it is mixed by pipetting and then oscillated at 4° C. for 30 min. Subsequently, it is centrifuged at 12,000 rpm at 4° C. for 10 min. The precipitate is the insoluble protein, and the supernatant is the soluble protein.
5. Two volumes of acetone are added to the supernatant SP from step 4, and precipitation is carried out at −20° C. for 1 hour. It is then centrifuged at 12,000 rpm at 4° C. for 10 min, the supernatant is discarded, and the precipitate is the soluble protein.
6. Corresponding amounts of PBS and SDS are added to the total protein, insoluble protein, and soluble protein (the biomass is uniformly adjusted to 6 according to the OD₆₀₀ value), and they are boiled in a water bath for 10 min. Protein expression is verified by SDS-PAGE.

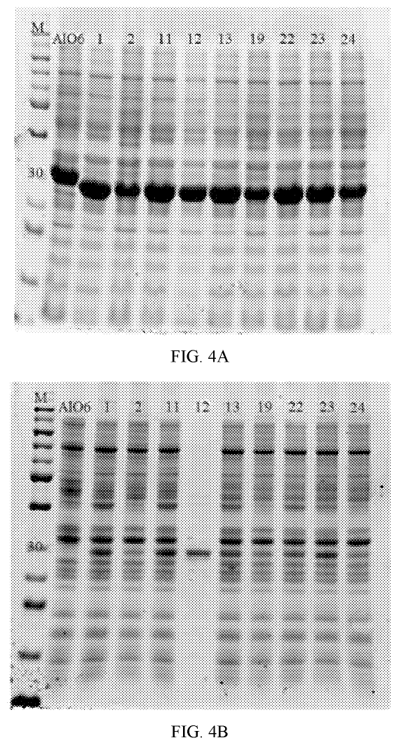

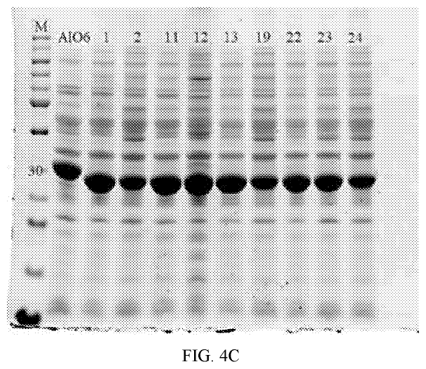

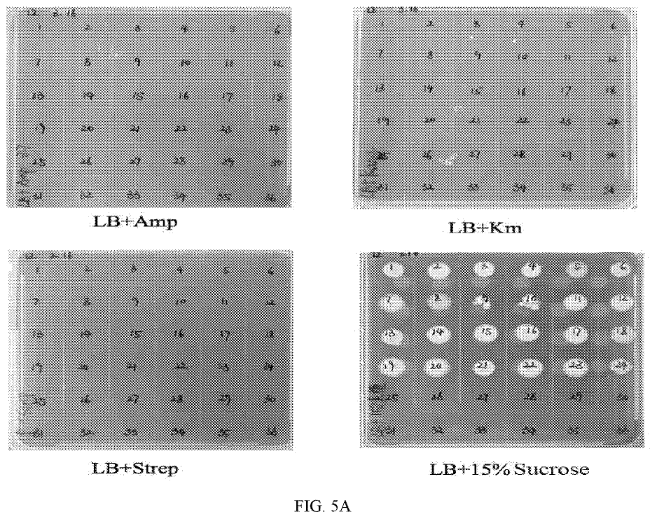

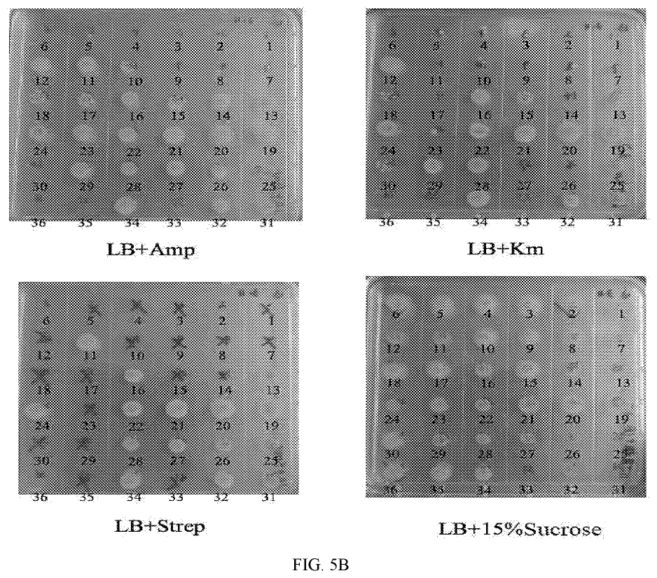

As can be seen from FIGS. 4A-4C, the size of the target protein AIO6 is approximately 32 kDa. Strains with good solubility and high expression levels (strains No. 1, No. 2, No. 11, No. 12, No. 13, No. 19, No. 22, No. 23, and No. 24) are selected for subsequent experiments.

### IV. Enzyme Activity Assay of Transposon Strains

1. The strains verified for protein expression (strains No. 1, No. 2, No. 11, No. 12, No. 13, No. 19, No. 22, No. 23, and No. 24) are selected and are inoculated into 20 mL of LB medium containing 100 μg/mL ampicillin, 50 μg/mL kanamycin, and 50 μg/mL streptomycin. They are cultured overnight at 37° C., and use a strain containing pET28a-AIO6 (kanamycin) as a control.
2. 500 μL of the culture from step 1 is transferred to 50 mL of LB medium containing the corresponding antibiotics, and cultured at 37° C. until the OD₆₀₀ reaches 0.6-0.8.
3. 24 μL of IPTG (final concentration of 0.4 mM) is added and induced at 16° C. for 16-18 hours.
4. 50 mL of the bacterial culture is centrifuged at 4000 rpm for 10 min to collect the precipitate. 10 mL of PBS is added to wash the bacteria, centrifuged at 4000 rpm for 10 min, the supernatant is discarded, 8 mL of PBS is added to resuspend the bacteria, and the resulting suspension is placed in a metal ice box for ultrasonic disruption for 10 min (sonicate for 2 s and cool for 3 s).
5. The sample is centrifuged at 12000 rpm at 4° C. for 10 min after sonication, and the supernatant is filtered through a 0.22 μm aqueous filter membrane.
6. HPLC Enzyme Activity Assay
a. Substrate preparation: the substrate stock solution (3-oxo-C8-HSL (Sigma-Aldrich, O1764-100), 5 mg/mL) is diluted to 0.5 mg/mL with PBS.
b. Protein dilution: the protein is diluted appropriately with PBS.
c. Reaction system: For the treatment group, the 400 μL system consists of 100 μL of substrate+100 μL of protein+200 μL of PBS; for the control group, the 400 μL system consists of 100 μL of substrate+200 μL of PBS+100 μL of protein (the corresponding protein is added to the control group after the reaction is terminated with methanol).
d. Reaction conditions: it is reacted in a 30° C. water bath for 15 min.
e. Reaction termination: 600 μL of methanol is added to terminate the enzyme activity reaction.

After reaction termination, the sample is mixed and it is filtered through a 0.22 μm organic filter membrane. Then, HPLC detection is performed under the following conditions: sample injection volume of 20 μL, wavelength of 254 nm, aqueous phase (800 mL of water with 300 μL of triethylamine):organic phase (acetonitrile)=0.64:0.36, and a flow rate of 1 mL/min.

The enzyme activity of the corresponding strains is calculated based on the HPLC detection data. The definition of enzyme activity is as follows: under the conditions of 30° C. and pH 6.5, the amount of enzyme required to hydrolyze 1 nmoL of N-acyl-homoserine lactone per minute from a 50 μg/mL N-acyl-homoserine lactone solution is defined as 1 unit of N-acyl-homoserine lactonase activity, denoted as U.

### Enzyme Activity Calculation Formula:

$$X = \frac{Δ C \times V \times n}{0.1 \times M \times t \times m} \times 1000$$

X: activity of N-acyl-homoserine lactonase in the enzyme dilution, U/g;
ΔC: change in substrate concentration, μg/mL;
V: dissolution volume of the sample, mL;
n: dilution factor of the sample;
0.1: volume of enzyme solution added to the total reaction system, mL;
M: molar mass of the substrate, g/mol, M=241.8;
t: reaction time of the enzyme, min;
1000: conversion factor.

The determination results of the two parallel samples are expressed as the arithmetic mean.

The enzyme activity assay results are shown in Table 3: the enzyme activities of most AIO6 transposon strains are significantly higher than that of the control group (pET28a-AIO6). Among them, strain 12 has an enzyme activity of 4560.9 U/mL/OD₆₀₀ and strain 13 has an enzyme activity of 4427.4 U/mL/OD₆₀₀, which are relatively high. These strains are proposed as candidate strains for subsequent screening.

TABLE 3
Enzyme Activity Assay Results of AIO6 Transposon Strains

| | Enzyme |
|---|---|
| Name | Activity(U/mL/OD₆₀₀) |
| pET28a-AIO6 | 1855.7 |
| 1 | 3505.9 |
| 2 | 1657.3 |
| 11 | 4028.2 |
| 12 | 4560.9 |
| 18 | 4427.4 |
| 19 | 1533.3 |
| 22 | 3949.0 |
| 23 | 3058.9 |
| 24 | 2073.8 |

### Example 2: Termination of Transposition by pCutamp

### 1. Acquisition of AIO6 Plasmid-Free Multi-Copy Integrated Strains

Based on the enzyme activity assay results and the number of transposition insertion sites from Example 1, strains 12 and 13, which exhibited the highest levels of transposition, are selected. Competent cells are prepared from these transposon strains (harboring three plasmids: pRE57I-AIO6, pTet-tns, and pTetQcas-8+IS186) according to the method for preparing competent Escherichia coli cells. The pCutamp vector is transformed into these competent cells via electroporation (1.85 k, 200 Ω, 25 μF), followed by recovery in 1 mL of SOC medium (Hopebio, HBDC002) at 37° C. for 1 hour. The cells are then collected by centrifugation and transferred to 4 mL of LB medium containing 50 μg/mL apramycin (Macklin, A899829-lg) and 10 mM rhamnose (Innochem, A65280), followed by shaking at 37° C. for 10 hours. Subsequently, 100 μL of the culture is inoculated onto LB agar plates containing 50 μg/mL apramycin and 10 mM rhamnose, and incubated overnight at 37° C.

Colonies grown on the LB plates are streaked onto four types of LB agar plates containing 100 μg/mL ampicillin, 50 μg/mL kanamycin, 50 μg/mL streptomycin, and 15% sucrose, respectively. Colonies that failed to grow on plates containing ampicillin, kanamycin, and streptomycin are considered to have eliminated pRE57I-AIO6, pTnsABC, and pQCascade-IS1.

The results, as shown in FIGS. 5A-5B, indicated that the transposon plasmids are eliminated from all colonies of strain 12, while three colonies (2, 6, and 36) of strain 13 had eliminated the transposon plasmids.

Subsequently, the corresponding colonies that grew only on the sucrose plates are re-inoculated onto LB agar plates without any antibiotics and LB plates containing apramycin to verify the curing of pCutamp. Colonies that failed to grow on plates containing apramycin are identified as plasmid-free multi-copy integrated strains.

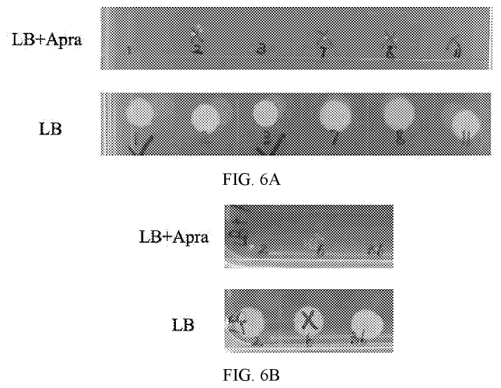

The results, as shown in FIGS. 6A-6B, indicated that clones 1 and 3 of strain 12 (i.e., 12-1 and 12-3), and clones 2 and 36 of strain 13 (i.e., 13-2 and 13-36) did not grow on plates containing apramycin. Ultimately, AIO6 plasmid-free multi-copy integrated strains 12-1, 12-3, 13-2, and 13-36 are obtained.

### 2. Induction and Expression of Plasmid-Free Multi-Copy Recombinant Strains

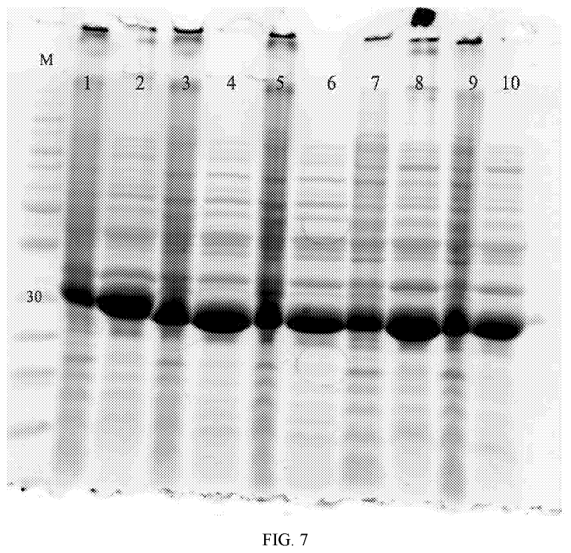

(1) Plasmid-free multi-copy recombinant strains 12-1, 12-3, 13-2, and 13-36 are inoculated into 20 mL of LB medium and cultured overnight at 37° C., with pET28a-AIO6 (kanamycin) used as a control.
(2) 500 μL of the culture from step (1) is transferred to 50 mL of LB medium and cultured at 37° C. until the OD₆₀₀ reached 0.6-0.8.
(3) 24 μL of IPTG (final concentration of 0.4 mM) is added, and induction is performed at 16° C. for 16-18 hours.
(4) Three 1 mL samples from step (3) are taken: one for measuring OD600 (200 μL; 4 replicates); one is centrifuged at 12000 rpm at 4° C., the supernatant is discarded, and the pellet (total protein, C) is stored at −20° C.; one is subjected to a soluble and insoluble protein experiment. The sample is centrifuged at 12000 rpm at 4° C., the supernatant is discarded, 0.5 mL of BugBuster is added to the pellet, mixed thoroughly, and shaken at 4° C. for 30 minutes. Subsequently, it is centrifuged at 12000 rpm at 4° C. for 10 minutes, with the pellet being the insoluble protein (IP) and the supernatant being the soluble protein (SP).
(5) Two volumes of acetone are added to the supernatant SP from step (4), and precipitation is performed at −20° C. for 1 hour. It is then centrifuged at 12000 rpm at 4° C. for 10 minutes, the supernatant is discarded, and the pellet is the soluble protein (SP).
(6) PBS and SDS are added to the total protein, insoluble protein, and soluble protein (the biomass is uniformly adjusted to 6 based on the OD₆₀₀ value), and the samples are boiled in a water bath for 10 minutes for protein expression verification. SDS-PAGE results showed that all selected colonies normally expressed the target protein N-acyl-homoserine lactonase AIO6 (FIG. 7).

### 3. Enzyme Activity Assay of Plasmid-Free Multi-Copy Recombinant Strains

The enzyme activity assay of plasmid-free multi-copy recombinant strains 12-1, 12-3, 13-2, and 13-36 is performed according to the method described in step four of Example 1, with Escherichia coli harboring the pET28a-AIO6 (kanamycin) vector used as the control strain.

The enzyme activity results are shown in Table 4: the enzyme activities of the plasmid-free multi-copy recombinant strains 12-1, 12-3, 13-2, and 13-36 obtained in this disclosure, which lacked antibiotic selection markers, are all higher than those of the pET28a-AIO6 strain selected through antibiotics.

TABLE 4
Enzyme Activity Assay Results of AIO6 Plasmid-Free
Multi-Copy Recombinant Strains

| | Enzyme |
|---|---|
| Name | Activity (U/mL/OD) |
| pET288-AIO6 | 2939.1 |
| 12-1 | 5866.3 |
| 12-3 | 3818.6 |
| 13-2 | 6148.9 |
| 13-36 | 6589.8 |

The present disclosure has been described in detail above. For those skilled in the art, within the scope and spirit of the present disclosure and without the need for unnecessary experimentation, the disclosure can be implemented over a wide range of equivalent parameters, concentrations, and conditions. Although specific embodiments of the disclosure have been presented, it should be understood that further improvements to the disclosure can be made. In summary, in accordance with the principles of the present disclosure, this application is intended to encompass any modifications, applications, or improvements to the disclosure, including changes made using conventional techniques known in the art that deviate from the scope already disclosed in this application.

What is claimed is:

1. A method for constructing a recombinant Escherichia coli without antibiotic resistance screening markers, comprising introducing a coding gene of N-acyl homoserine lactonase AIO6 into a recipient Escherichia coli, followed by screening without antibiotic resistance markers to obtain the recombinant Escherichia coli, wherein the introducing the coding gene of the N-acyl homoserine lactonase AIO6 into the recipient Escherichia coli comprises the following steps:
1) introducing a vector containing the N-acyl homoserine lactonase AIO6 and a vector containing coding genes of TnsA, TnsB, and TnsC proteins into the recipient Escherichia coli, and culturing to obtain a first transformant;
2) introducing a multi-target gene editing plasmid into the first transformant in the step 1), and culturing to obtain a second transformant;
3) conducting primary screening of the second transformant in the step 2) to select a strain with a high transposition efficiency and/or a high enzyme activity of the N-acyl homoserine lactonase AIO6; and
4) introducing a pCutamp vector into the strain to stop a transposition process, and screening using an antibiotic plate to obtain strains, wherein the strains grow on a rhamnose medium but fail to grow on the antibiotic plate, wherein the strains are the recombinant Escherichia coli without the antibiotic resistance screening markers.

2. The method according to claim 1, wherein the amino acid sequence of the N-acyl homoserine lactonase AIO6 is SEQ ID NO: 1, and the nucleotide sequence of the coding gene of the N-acyl homoserine lactonase AIO6 is SEQ ID NO: 2.

3. The method according to claim 1, wherein the multi-target gene editing plasmid is pTetQcas-8+IS186.

4. A method for using the method according to claim 1 in one of the following applications:
producing N-acyl homoserine lactonase;
increasing a yield of the N-acyl homoserine lactonase; and
preparing a recombinant strain producing the N-acyl homoserine lactonase without the antibiotic resistance screening markers.

5. A recombinant Escherichia coli constructed by the method according to claim 1.

6. A method for using the recombinant Escherichia coli according to claim 5 in one of the following applications:
producing N-acyl homoserine lactonase; and
increasing a yield of the N-acyl homoserine lactonase.

7. A method for producing N-acyl homoserine lactonase, comprising culturing the recombinant Escherichia coli according to claim 5 to obtain a fermentation product; and obtaining the N-acyl homoserine lactonase from the fermentation product.

8. The method according to claim 2, wherein the multi-target gene editing plasmid is pTetQcas-8+IS186.

9. The method according to claim 4, wherein in the method for constructing the recombinant Escherichia coli without the antibiotic resistance screening markers, the amino acid sequence of the N-acyl homoserine lactonase AIO6 is SEQ ID NO: 1, and the nucleotide sequence of the coding gene of the N-acyl homoserine lactonase AIO6 is SEQ ID NO: 2.

10. The recombinant Escherichia coli according to claim 5, wherein in the method, the amino acid sequence of the N-acyl homoserine lactonase AIO6 is SEQ ID NO: 1, and the nucleotide sequence of the coding gene of the N-acyl homoserine lactonase AIO6 is SEQ ID NO: 2.

11. The recombinant Escherichia coli according to claim 5, wherein in the method, the multi-target gene editing plasmid is pTetQcas-8+IS186.
